(19) United States
(12) Patent Application Publication
(10) Pub. No.: US 2007/0098376 A1
(43) Pub. Date: May 3, 2007

(54) Vapour dispersion

(75) Inventors: Colin Brown, Bracknell Berkshire (GB); Guy Edward Naish, Bicester Oxfordshire (GB)

Correspondence Address: NORRIS, MCLAUGHLIN & MARCUS, 875 THIRD AVE, 18TH FLOOR, NEW YORK, NY, 10022, US

(73) Assignee: Givaudan SA, Vernier (CH)

(21) Appl. No.: 10/561,753

(22) PCT Filed: Jun. 17, 2004
(86) PCT No.: PCT/CH04/00396

§ 371 Date: Feb. 1, 2006

(51) Int. Cl. A61L 9/00; A61L 9/03

(52) U.S. Cl. 392/386; 422/5

(57) ABSTRACT

An apparatus adapted to transmit an active substance in the vapour phase to an atmosphere, comprising an active substance incorporated in a sublimable carrier substance (12) and a heating element (4) that interfaces with only part of the total sublimable carrier substance, carrier substance and heating element being so arranged that the sublimable carrier substance subliming at the heating element/sublimable carrier substance interface is replaced by further sublimable carrier substance. The apparatus is not prone to spillage, as are current volatile liquid-based apparatus. It also allows the easy observation of the approaching end of life of the carrier substance/active substance and its need for replacement, so that continuity of transmission is assured.

This invention relates to the use of sublimable materials to transmit substances in the vapour phase into an atmosphere and to apparatus for achieving this.

It is often desired to transmit into an atmosphere an active substance, such as a fragrance or an insecticide, in the vapour phase. One well-known way of doing this is by incorporating the active substance into a volatile liquid carrier, which is then evaporated from an evaporating surface, typically a porous wick, optionally with the assistance of heat and/or forced ventilation. Such systems are widely used and have enjoyed considerable success. However, they can also have drawbacks. One such drawback is the ease of spillage of the volatile liquid. This can be overcome, for example, by capturing the active substance and carrier in a gel. This in turn has the problem that there is no clear indication of the end of the life of the system (the gel remains when the active substance is exhausted). This means that it is hard to see when replacement is needed, in order for transmission of the active substance to continue.

It has been suggested that an alternative means of transmitting an active substance in the vapour phase into an atmosphere is by means of a carrier substance that sublimes, that is, that goes directly from solid phase to gaseous phase at normal temperature and pressure, without passing through a liquid phase. This would have the advantage of providing good end-of-life indication, as the material is easily visible. Systems utilising sublimable materials have been suggested, but none has ever found commercial acceptance. Part of the problem is that, for practical purposes, heating is required. Heaters are known from prior use in volatile liquid systems, but these have not worked efficiently with sublimable materials.

It has now been found that a combination of a sublimable material and a particular variety of heating can overcome these disadvantages and permit the effective utilisation of the inherent advantages of sublimable materials. The invention therefore provides an apparatus adapted to transmit an active substance in the vapour phase to an atmosphere, comprising an active substance incorporated in a sublimable carrier substance and a heating element that has a subliming effect on only part of the total sublimable carrier substance, carrier substance and heating element being arranged such that the sublimable carrier substance subliming at that part of the sublimable carrier substance affected by the heating element is replaced by further sublimable carrier substance.

The invention further provides a method of transmitting an active substance in the vapour phase into an atmosphere by incorporating the active substance into a sublimable carrier substance and heating the carrier substance by means of a heating element, the heating element having a subliming effect on only part of the carrier substance and the carrier substance being disposed with respect to the heating element such that carrier substance that sublimes is replaced by further carrier substance.

The active substance may be any such substance whose presence in an atmosphere is desired. For example, it may be a fragrance, a deodorant, an insecticide or other substance whose transmission into an atmosphere is desired. The following lists of active substances are purely exemplary and do not restrict the choice of active materials in any way.

By way of non-limiting example fragrances useful in the present invention include one or more materials selected from natural products such as essential oils, absolutes, resinoids, resins, concretes, and synthetic perfume components such as hydrocarbons, alcohols, aldehydes, ketones, ethers, acids, acetals, ketals and nitriles, including saturated and unsaturated compounds, aliphatic, carbocyclic and heterocyclic compounds. Examples of preferred fragrance components are any of those fragrances selected from Aldron, Ambrettolide, Ambroxan, Benzyl Cinnamate, Benzyl Salicylate, Boisambrene Forte, Cedrol Crystals, Cedryl Acetate Crystals, Celestolide/Crysolide, Cetalox, Citronellyl Ethoxalate, Fixal, Fixolide, Galaxolide 50 Dep, Guaiacwood Acetate, Cis-3-Hexenyl Salicylate, Hexyl Cinnamic Aldehyde, Hexyl Salicylate, Iso E Super, Linalyl Benzoate Fcc, Linalyl Cinnamate, Linalyl Phenyl Acetate, Methyl Cedryl Ketone, Musk Cpd, Musk Ketone, Musk Tibetine, Musk Xylol, Myraldyl Acetate, Nerolidyl Acetate, Novalide, Okoumal, Para Cresyl Caprylate, Para Cresyl Phenyl Acetate Crystals, Phantolid Crystals, Phenyl Ethyl Cinnamate, Phenyl Ethyl Salicylate, Rosone, Sandela (Lin), Tetradecanitrile, Thibetolide, Traseolide 100, Trimofix O,2-Methyl Pyrazine, Acetaldehyde phenylethyl propyl acetal, Acetophenone, Alcohol C6 (in the following the notation Cn comprises all substances having n carbon atoms and one hydroxyl function), Alcohol C8, Aldehyde C6 (in the following the notation Cn encompasses all isomers having n carbon atoms and one aldehyde function), Aldehyde C7, Aldehyde C8, Aldehyde C9, Nonenylic Aldhyde, Allyl Amyl Glycolate, Allyl Caproate, Amyl Butyrate, Aldehyde Anisique, Benzaldehyde, Benzyl Acetate, Benzyl Acetone, Benzyl Alcohol, Benzyl Butyrate, Benzyl Formate, Benzyl Iso Valerate, Benzyl Methyl Ether, Benzyl Propionate, Bergamyl Acetate, Butyl Acetate, Camphor, 3-Methyl-5-propyl-2-cyclohexenone, Cinnamic Aldehyde, Cis-3-Hexenol, Cis-3-Hexenyl Acetate, Cis-3-Hexenyl Formate, Cis-3-Hexenyl Iso Butyrate, Cis-3-Hexenyl Propionate, Cis-3-Hexenyl Tiglate, Citronellal, Citronellol, Citronellyl Nitrile, 2-Hydroxy-3-methyl-2-Cyclopenten-1-one, Cuminic Aldehyde, Cyclal C, Acetic Acid (cycloheyloxy)-2-propenylester, Damascenone, Damascone Alpha, Damascone Beta, Diethyl Malonate, Dihydro Jasmone, Dihydro Linalool, Dihydro Myrcenol, Dihydro Terpineol, Dimethyl Anthranilate, Dimethyl Benzyl Carbinol, Dimethyl Benzyl Carbinyl Acetate, Dimethyl Octenone, Dimetol, Dimyrcetol, Estragole, Ethyl Acetate, Ethyl Aceto Acetate, Ethyl Benzoate, Ethyl Heptoate, Ethyl Linalool, Ethyl Salicylate, Ethyl-2-Methyl Butyrate, Eucalyptol, Eugenol, Fenchyl Acetate, Fenchyl Alcohol, 4-Phenyl-2,4,6-trimethyl 1,3-dioxane, Methyl 2-octynoate, 4-Isopropylcyclohexanol, 2-sec-Butylcyclohexanone, Styralyl acetate, Geranyl nitrile, Hexyl Acetate, Ionone Alpha, Iso Amyl Acetate, Iso Butyl Acetate, Iso Cyclo Citral, Dihydroisojasmone, Iso Menthone, Iso Pentyrate, Iso Pulegol, cis-Jasmone, Laevo Carvone, Phenylacetaldehyde glycerylacetal, carbinic acid 3-Hexenyl Methyl Ether, 1-Methyl-cyclohexa-1,3-diene, Linalool, Linalool Oxide, 2,6-Dimethyl-5-heptenal, Menthol, Menthone, Methyl Acetophenone, Methyl Amyl Ketone, Methyl Benzoate, Methyl Cinnamic Aldehyde Alpha, Methyl Heptenone, Methyl Hexyl Ketone, Methyl Para Cresol, Methyl Phenyl Acetate, Methyl Salicylate, Neral, Nerol, 4-tert-Pentyl-cyclohexanone, Para Cresol, Para Cresyl Acetate, Para Tertiary Butyl Cyclohexanone, Para Tolyl Aldehyde, Phenyl Acetaldehyde, Phenyl Ethyl Acetate, Phenyl ethyl alcohol, Phenyl ethyl butyrate, Phenyl ethyl formate, Phenyl ethyl iso butyrate, Phenyl ethyl propionate, Phenyl Propyl Acetate, Phenyl Propyl Aldehyde, Tetrahydro-2,4-dimethyl-4-pentyl-furan, 4-Methyl-2-(2-methyl-1-propenyl)tetrahydropyran, 5-Methyl-3-heptanone oxime, Styralyl Propionate, Styrene Monomer, 4-Methylphenylacetaldehyde, Terpineol, Terpinolene, Tetrahydro Linalool, Tetrahydro Myrcenol, Trans-2-Hexenal, 4,7-Methano-1H-3A,4,5,6,7,7A-hexahydro-acetate and Viridine.

Perfumes that may be used include any material which provides a fragrancing effect, including the fragrance materials recited above.

Insecticides or other insect control compositions useful in the present invention include one or more materials selected from such as citronella oil, tolu and Peru balsams, camphor, as well as other insecticides or other insect control compositions known to the art.

Sanitizing agents useful in the present invention include one or more materials selected from alcohols, glycols as well as other volatile sanitizing agents known to the art.

These and other suitable materials which may be useful may be commercially obtained from a variety of suppliers including: Givaudan Corp. (Teaneck, N.J.); Berje Inc. (Bloomfield, N.J.); BBA Aroma Chemical Div. of Union Camp Corp. (Wayne, N.J.); Firmenich Inc. (Plainsboro N.J.); Quest International Fragrances Inc. (Mt. Olive Township, N.J.); Robertet Fragrances Inc. (Oakland, N.J.).

The active substance may be provided in the form of a neat composition, or as an aqueous mixture, an organic mixture or an aqueous-organic mixture, which may include of one or more volatile or evaporable materials.

The sublimable carrier substance may be any such substance whose presence in the target atmosphere is innocuous. What may be considered as “innocuous” may vary considerably, depending on the nature of the use and the atmosphere—what may be considered innocuous in one location may not be thus considered in another. However, the skilled person can easily ascertain what is innocuous for any given application.

Typical examples of sublimable carrier substances include adamantane and derivatives thereof, para-dichlorobenzene, 2,4,6-triisopropyl-1,3,5-trioxane, camphor, naphthalene, 1,2,3-trichlorobenzene, 1,4-cyclohexanediol, menthol, acetamide, diphenylethane, hexachloroethane, benzoid acid, benzophenone, benzyl cinnamate, benzyl isoeugenol, benzylidene acetone, cedrol, cinnamic alcohol, coumarin, dimethyl fumarate, hydroquinone dimethyl ether, acetyl isoeugenol, methyl cinnamate, methyl coumarin, napthyl ethyl ether, phthalide, thymol. Other factors governing the choice of the sublimable solid material includes the type of indicator to be used, the desired lifetime, the exposed surface area of the sublimable solid material, as well as other factors which may become apparent from examining the performance characteristics of a particular volatile substance dispenser produced in accordance with the present inventive teaching. For example, when it is desired that the sublimable carrier material have little or no discernible odour, for example, when the device is used to deliver a fragrance or a perfume, a sublimable carrier material that has a noticeable scent, such as para-dichlorobenzene, camphor, and naphthalene, should be avoided as it may undesirably affect the consumer's perception of the fragrance or perfume being delivered.

Particularly useful and particularly preferred for use in the sublimable solid material are one or more materials selected from the group consisting of: camphor, 1,4-cyclohexanediol, tetrahydrodicyclopentadiene, and adamantane and derivatives thereof. Adamantane is particularly preferred. Further particularly preferred sublimable solid materials include adamantane derivatives as well as salts thereof. By way of non-limiting example, useful derivatives of adamantane include: 1-acetoamido-adamantane, 1-adamantanecarboxylic acid, 1-aminoadamantane hydrochloride, 1-amino-adamantane sulfate, 1-hydroxyadamantane, 1,3-dimethyladamantane, 1-chloro-3,5-dimethyl-adamantane, adamantylmethlketone, 2-adamantanone, 2-methyl-2-adamantanol, 2-ethyl-2-adamantanol, and diamantane.

The sublimable carrier substance or mixture of such substances should be one that sublimes over time when maintained within the temperature range of about 0° C. to about 30° C., preferably when maintained within the temperature range of about 5° C. to about 30° C. at normal atmospheric pressure.

For the purposes of this invention, particularly preferred sublimable carrier substances are adamantane, cyclododecane and tetrahydrodicyclopentadiene and mixtures thereof.

The active substance may be incorporated into the sublimable carrier substance by any convenient means, for example by first mixing together the sublimable carrier substances (if more than one is used) and then thoroughly mixing in the active substance. The carrier substance/active substance combination is preferably in a solid compressed mass. It may also be used in granular or powder form, but this is less convenient in use.

The sublimable carrier and the heating element are preferably provided with a support body whose function it is to maintain the two in a position relative to one another such that they can function properly and reliably. The support body also provides the conduit for the energy required to sublime the carrier. This is generally electrical wiring leading to the heating element. The form of the support body is not critical and it can be any suitable decorative or utilitarian shape that allows it to carry out its function.

The heating element may be any suitable heating element that has a subliming effect on only part of the sublimable carrier substance. It is preferably an electrical heating element. It may cause the sublimable carrier substance to sublime by radiant energy, that is, by the element not being in physical contact with the carrier substance and radiating its heat to the carrier substance. However, it is more efficient to have physical contact between heating element and carrier substance and this is preferred. Further preferred embodiments will be described hereinunder.

The heating element and the sublimable carrier substance are disposed in relation to each other so that the sublimable carrier substance subliming in the vicinity of the heating element is replaced by further carrier substance. This can be achieved by any convenient means. For example, it may be achieved by mechanical or electrical means that causes more carrier substance to move into the vicinity of the heating element. However, in general, for simplicity and practicality, it is preferred that the heating element be located below the sublimable carrier substance and continuous feeding of sublimable carrier substance to replace the subliming carrier substance in the vicinity of the heating element be achieved by gravity. Thus, for example, the heating element may be an element that is placed at the bottom of a cylindrical solid of sublimable carrier substance, and as the carrier substance nearest the heating element sublimes under the heat of the element, its place is taken by unsublimed solid immediately above it. Similarly, if the sublimable carrier substance is in granular or powder form, it may be housed in a tube mounted above a heating element and it is constrained to move towards the element as that carrier substance interfacing with the element sublimes. This works best when the heating element makes physical contact with the carrier substance, and more particularly when the carrier substance rests on a heated surface.

An especially useful heating element for use in this last-mentioned embodiment is an electrical heating element that is deposited on a surface on which the heated surface rests (hereinafter “surface heating element”). This can be achieved by any suitable means, but again the simplest is the most preferred, namely the deposition of the element on a flat surface that is positioned in a horizontal plane below the sublimable carrier substance, with which it has contact. As the carrier substance sublimes, more carrier substance moves down to take its place. A typical heating element of this type consists of a heating circuit of PTC (positive temperature coefficient) resistor paste screen-printed on to a flat substrate such as a 96% alumina plate or porcelain enamelled steel.

In a preferred embodiment, the carrier substance is located within a tube mounted above the surface heating element, which tube constrains the carrier substance. Preferably the tube is at least partially transparent or sufficiently translucent to allow visual inspection, so that the use rate of the carrier substance may be observed and a replacement provided prior to its complete exhaustion. This tube may be supplied as part of a refill, which can be easily fitted into and removed from a heating unit, which may be adapted to plug directly into an electrical power point.

The refill as mentioned hereinabove is preferably of the form of a tube that is completely sealed except for a vent at the lower end (adjacent to the heating element when the refill is in place) to permit the escape of sublimed material. The lower end may be sealed by a conductive material, such as metal foil, so that, in position, the carrier substance rests on this foil and the heat of the heating element mounted in the support body is communicated through to the sublimable carrier in the tube. In a more preferred embodiment, the surface bearing the heating element itself forms the lower end of the tube and the support body provides only the wiring and electrical contacts for the heating element. The invention therefore additionally provides an apparatus adapted to transmit in the vapour phase an active substance into an atmosphere, the apparatus comprising a support body and a replaceable active substance reservoir, the active substance being incorporated in a sublimable carrier substance, which carrier substance is contained within a tube adapted to be mounted substantially vertically in the support body, the tube being completely sealed except for a vent at the lower end adapted to permit release of sublimed material, and the sealing of the lower end being accomplished by a surface bearing an electrical heating element, the support body comprising suitable electrical wiring and connections for the supply of heating electricity to the element.

The surface heating element has the considerable advantage that it can be made precisely the right size for any given application. It is also more efficient that other possible heaters, and it can be easily tailored to the correct temperature for a given active substance/sublimable carrier. It has the further advantage of being very safe, as it can be completely enclosed. The embodiment hereinabove described, in which a replaceable refill has incorporated therein a heating element, is especially safe, in that the removal of the refill for replacement also removes the hot element.

There are many possibilities and configurations, all within the scope of the invention. For example, it is possible to use two or more different surface heating elements, each tailored to release a different active ingredient. For example, in the case of fragrances, such an apparatus could be programmed, such that different fragrances are released at different times. In another embodiment, a tube constraining a sublimable carrier need not be vertical, but may be inclined to the vertical. If a tube is closed except for a vent at the bottom to allow the sublimed material to escape into the atmosphere, a portion of the sublimable material may rise vertically up the tube and condense on the walls and roof of the tube, making this material of no effect and possibly blocking the view of the sublimable carrier. A suitably inclined tube, with the vent on the upper side of the incline, can substantially or completely overcome this problem.

The apparatus of this invention have a number of advantages over known apparatus. Not only do they effectively transmit active substances into an atmosphere, but they also are essentially free of the danger of spilling and they can be easily configured so that their impending exhaustion can easily be seen and a replacement provided, without loss of effect. The preferred embodiments hereinabove described bring the other advantages already described of improved safety and efficiency.

The apparatus is now further described with reference to the drawings, which depict preferred embodiments and which are not intended to be limiting on the scope of the invention in any way.

FIG. 1 is a schematic longitudinal cross-section of an apparatus according to the invention, with refill not present.

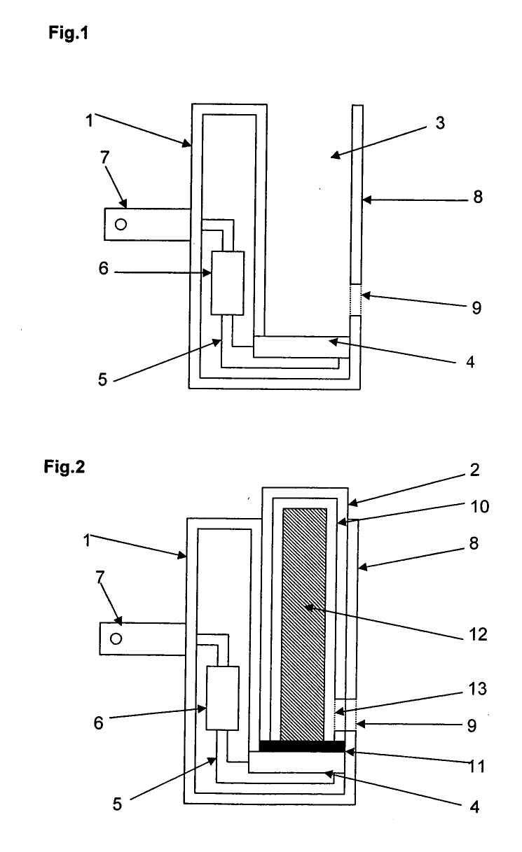

FIG. 2 is a schematic longitudinal cross-section of the apparatus of FIG. 1, with refill in place.

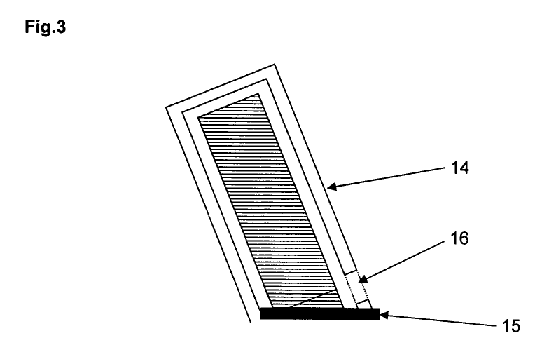

FIG. 3 is a schematic longitudinal cross-section of a refill of a different embodiment of the invention.

In FIGS. 1 and 2, the apparatus consists basically of a support body 1 and a refill 2. The particular support body illustrated is basically rectilinear, but the shape is not restricted in any way and can be in any suitable practical or decorative form. The support body comprises a recess 3, into which the refill 2 is removably fitted. At the bottom of this recess is a planar electrical heating element 4, consisting of a flat alumina plate, on which has been screen printed a heating circuit of PTC resistor paste. This circuit is connected by wires 5 to a control circuit 6 that contains a step-down transformer and may include other functional circuitry, such as a timing device for controlled release. This control unit is supplied with mains electricity via electrical connecting pins 7 that fit into a wall socket. The wiring and control circuit are housed within the support body and the connecting pins are integral with the support body. The front panel 8 of the support body is sufficiently transparent such that the refill, and therefore the quantity of sublimable carrier remaining, can be seen and the need for a replacement refill therefore assessed. The front panel additionally has a port 9, which allows the sublimable material to escape.

The refill 2 consists of a transparent plastic tube 10, open at that end that will be adjacent to the heating element. This open end is sealed by metal foil 11, which, when the refill is in place on the apparatus, sits on the heating element 4. Within the tube is a stick of sublimable carrier 12 that incorporates an active substance destined for release into an atmosphere. The tube 10 has a vent 13, located at its lower end, so that it matches the port 9 in the front panel 8 and permits release of sublimed material into the atmosphere. The port 13 is covered by a foil strip for transportation and storage, and this strip is removed immediately prior to placing the refill in the support body. Refill and support body are configured so that the refill is held firmly within the support body but it easily removed for replacement.

In use, the apparatus is simply plugged into a suitable mains electrical outlet. This will supply current to the heating element 4, which in turn will heat the foil 11, which will cause that part of the sublimable material 12 resting against the foil to sublime. This sublimed material is released into the atmosphere through aligned ports 9 and 13.

In FIG. 3, the refill is configured so that the tube 14 is inclined away from the perpendicular with respect to the plane of the foil 15. When the refill is in place on a suitable support body, the port 16 for release of sublimed material is located on the upper side of this tube. This substantially avoids any tendency of the sublimed material to rise up and condense in the interior of the tube of the refill, rather than go into the atmosphere.

1. An apparatus adapted to transmit an active substance in the vapour phase to an atmosphere, comprising an active substance incorporated in a sublimable carrier substance (12) and a heating element (4) that has a subliming effect on only part of the total sublimable carrier substance, carrier substance and heating element being arranged such that the sublimable carrier substance subliming at that part of the sublimable carrier substance affected by the heating element is replaced by further sublimable carrier substance.

2. An apparatus according to claim 1, in which the heating element (4) applies heat to the carrier substance by physical contact with the carrier substance.

3. An apparatus according to claim 1 wherein the carrier substance rests on a substantially horizontal surface, which is heated by the heating element.

4. An apparatus according to claim 1 wherein the substantially horizontal surface is the heating element (4).

5. An apparatus according to claim 4, in which the carrier substance forms part of a replaceable refill (2).

6. An apparatus according to claim 1, in which the carrier substance (12) is contained within a tube (10) adapted to be mounted substantially vertically in a support body (1), the tube being completely sealed except for a vent (13) at a lower end thereof, adapted to permit release of sublimed material, the sealing of the lower end being accomplished by a surface that is substantially horizontal and that is in intimate, heat-conducting contact with an electrical heating element (4), the support body comprising suitable electrical wiring (5) and connections (7) for the supply of heating electricity to the element.

7. A method of transmitting an active substance in the vapour phase into an atmosphere by incorporating the active substance into a sublimable carrier substance (12) and heating the carrier substance by means of a heating element (4) with which it has contact, the carrier substance being arranged with respect to the heating element such that carrier substance that sublimes at the contact between heating element and carrier substance is replaced by further carrier substance.

8. An apparatus according to claim 2 wherein the carrier substance rests on a substantially horizontal surface, which is heated by the heating element.

9. An apparatus according to claim 2 substantially horizontal surface is the heating element (4).

10. An apparatus according to claim 9, in which the carrier substance forms part of a replaceable refill (2).

11. An apparatus according to claim 3 wherein the substantially horizontal surface is the heating element (4).

12. An apparatus according to claim 11, in which the carrier substance forms part of a replaceable refill (2).
